(19) United States
(12) Patent Application Publication
(10) Pub. No.: US 2026/0114784 A1
(43) Pub. Date: Apr. 30, 2026

(54) SELECTIVELY FILTERING NOISE FROM CARDIAC SIGNALS

(71) Applicant: Medtronic, Inc., Minneapolis, MN (US)

(72) Inventors: Eric A. Schilling, Ham Lake, MN (US); Chad A. Bounds, Walnut Creek, CA (US); Xusheng Zhang, Shoreview, MN (US)

(21) Appl. No.: 19/112,503

(22) PCT Filed: Sep. 13, 2023
(86) PCT No.: PCT/IB2023/059098

§ 371 Date: Mar. 17, 2025

Related U.S. Application Data
(60) Provisional application No. 63/376,691, filed on Sep. 22, 2022.

(51) Int. Cl. A61B 5/363; A61B 5/00; A61B 5/352; A61N 1/05; A61N 1/362; A61N 1/365; A61N 1/375; A61N 1/39

(52) U.S. Cl. CPC A61B 5/363; A61B 5/352; A61B 5/4836; A61B 5/686; A61B 5/7217; A61N 1/0563; A61N 1/3621; A61N 1/365; A61N 1/3756; A61N 1/3925

(57) ABSTRACT

An example medical device includes a memory; and processing circuitry coupled to the memory, the processing circuitry is configured to: receive, from one or more electrodes coupled to the medical device, a cardiac signal; determine a risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activate a filter to filter the noise from the cardiac signal.

This application claims the benefit of U.S. Provisional Patent Application Ser. No. 63/376,691, filed Sep. 22, 2022, the entire content of which is incorporated herein by reference.

### TECHNICAL FIELD

The present application relates to an implantable medical device and, more particularly, implantable cardioverter defibrillators.

### BACKGROUND

Sudden cardiac death (SCD) affects more than 300,000 patients annually and accounts for nearly 6% of the annual mortality in the U.S., the vast majority due to fatal arrhythmias. Implantable cardioverter defibrillators (ICDs) are used to prevent SCD by applying low voltage electrical stimulation (e.g., anti-tachycardia pacing [ATP]) and/or a high voltage (e.g., defibrillation) electrical shock to the heart when a life-threatening arrhythmia is detected. Endocardial delivery of ATP may minimize patient pain and discomfort by reducing the number of defibrillation shocks delivered.

Implantation of transvenous leads for intracardiac pacing and defibrillation is not always possible due to anatomic anomalies, occluded vessels, or other issues. For these patients, an extravascular ICD (EV-ICD) system may provide an opportunity to receive life-saving implantable pacing/defibrillation systems. An EV-ICD system may include an extravascular lead that may be implanted in the substernal space and a device implanted on the thorax.

### SUMMARY

Episodes of noise, such as electromagnetic interference (EMI), may cause false tachyarrhythmia detections and result in subsequent unnecessary delivery of shocks or other therapeutic electrical signals to patients with ICDs and/or inhibition of pacing therapy. In addition, some ICDs may not filter noise signals because the filters have substantial current drain that may decrease the longevity of the ICD, reduce sensitivity of sensing and detection by the ICD, and/or have conflicting feature interactions with other devices.

The techniques of this disclosure are directed to selectively filtering noise, such as EMI, from cardiac signals without compromising detection sensitivity while at the same time minimizing the decrease in longevity of the ICD and conflicting feature interactions with other devices. In accordance with the techniques of the disclosure, an ICD may be configured to enable the noise filter(s), such as a notch filter, when needed or when more likely to be needed and disable the noise filter(s) when no longer needed or unlikely to be needed. Accordingly, the techniques described herein may help mitigate the potential for increase in inappropriate shock rate for ICDs that are susceptible to noise while also minimizing current drain by active filter(s) to minimize a potential decrease in longevity of the ICD and minimizing conflicting interactions with other devices.

In one example, this disclosure describes a medical device comprising a memory; and processing circuitry coupled to the memory, the processing circuitry is configured to: receive, from one or more electrodes coupled to the medical device, a cardiac signal; determine a risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activate a filter to filter the noise from the cardiac signal.

In another example, this disclosure describes a medical device system comprising a medical device configured to sense a cardiac signal with a sensor; and processing circuitry configured to: determine a risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activate a filter to filter the noise from the cardiac signal.

In another example, this disclosure describes a method comprising receiving, from one or more electrodes coupled to a medical device, a cardiac signal; determining, by processing circuitry, risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activating, by the processing circuitry, a filter to filter the noise from the cardiac signal.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1A is a front view of a patient with an extravascular ICD system implanted intra-thoracically.

FIG. 1B is a side view of the patient with the extravascular ICD system implanted intra-thoracically.

FIG. 1C is a transverse view of the patient with the extravascular ICD system implanted intra-thoracically.

FIG. 2 is a functional block diagram of an example configuration of electronic components of an example ICD.

FIG. 3 is a flow diagram illustrating an example method that may be performed by one or more medical devices to selectively activate a filter, in accordance with one or more techniques disclosed herein.

### DETAILED DESCRIPTION

Oversensing may cause painful inappropriate shocks in patients with implantable cardioverter defibrillators (ICDs). Leads for ICDs, such as right ventricular (RV) leads, substernal leads, subcutaneous leads, or other extra-cardiovascular leads, are susceptible to picking up noise and/or interference, such as electromagnetic interference (EMI). In some examples, an integrated bipolar lead, e.g., in which a coil electrode or portion thereof is used as one of the electrodes of a bipolar sensing pair, may be more susceptible to noise and interference than a true bipolar lead because the distance between a tip electrode and a ring or sense electrode is greater in an integrated bipolar lead than in a true bipolar lead. For example, episodes of EMI may cause false detections of ventricular fibrillation (VF) and result in subsequent unnecessary delivery of shocks or other therapeutic electrical signals to patients with ICDs and/or inhibition of pacing therapy. However, some ICDs may not reject EMI signals due to the need of high sensitivity for tachyarrhythmia sensing and detection. In some examples, a source of EMI a patient may encounter is from alternating current (AC) power sources. Some examples of frequencies of the EMI from those AC sources are 50 hertz (Hz) to 60 Hz. In some examples, these EMI frequencies may be close enough to the band-pass range to be sensed after attenuation by device sensing amplifiers, which may be designed to reject higher frequency EMI sources in the modern environment. In addition, some ICDs may not reject EMI signals because filters have intensive current drain which negatively impacts battery life that may decrease the longevity of the ICD. In addition, filters may reduce sensitivity of sensing and detection by the ICD, and/or have conflicting feature interactions with other devices.

In general, this disclosure is directed to techniques that facilitate avoidance of oversensing due to noise without compromising detection sensitivity or battery life of the ICD. In accordance with the techniques of the disclosure, an ICD may be configured to selectively enable the noise filter(s), such as a notch filter, when needed or when more likely to be needed and disable the noise filter(s) when no longer needed or unlikely to be needed. Accordingly, the techniques described herein may help mitigate the potential for increase in inappropriate shock rate for ICDs that are susceptible to noise while also minimizing current drain due to active filters to minimize the decrease in longevity of the ICD and minimizing conflicting feature interactions with some devices.

Although the noise detection techniques of this disclosure are primarily described herein with respect to detecting EMI, these techniques may be utilized to detect other types of noise. For example, the noise detection techniques of this disclosure my detect noise due to muscle or other motion artifacts, lead fractures or disconnections, magnetic resonance imaging, and other non-physiological noise. In some examples, the techniques described herein may be applied for detection of noise in ventricular electrograms to avoid inappropriate detection of ventricular tachyarrhythmia, e.g., ventricular tachycardia (VT) or VF, atrial electrograms to avoid inappropriate detection of atrial tachyarrhythmia, e.g., atrial tachycardia, fibrillation, or flutter, and/or for detection of noise in surface electrocardiograms.

As used herein, relational terms, such as “first” and “second,” “over” and “under,” “front” and “rear,” and the like, may be used solely to distinguish one entity or element from another entity or element without necessarily requiring or implying any physical or logical relationship or order between such entities or elements.

Although the techniques of this disclosure is primarily described herein with respect to extravascular ICD system, these techniques may be utilized by a transvenous ICD system or any ICD system, a leadless pacemaker, such as the Micra™ leadless pacemaker, available from Medtronic plc, or any pacemaker, or any device that senses cardiac or other physiological signals that may be impacted by EMI or other noise sources.

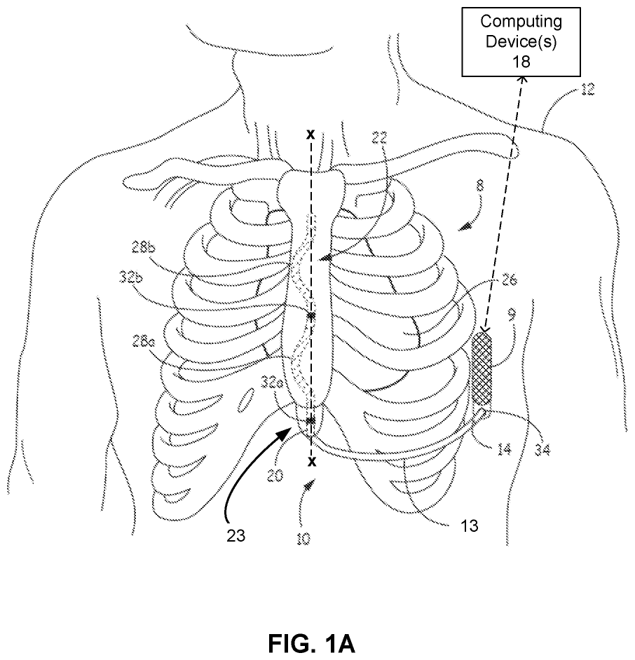

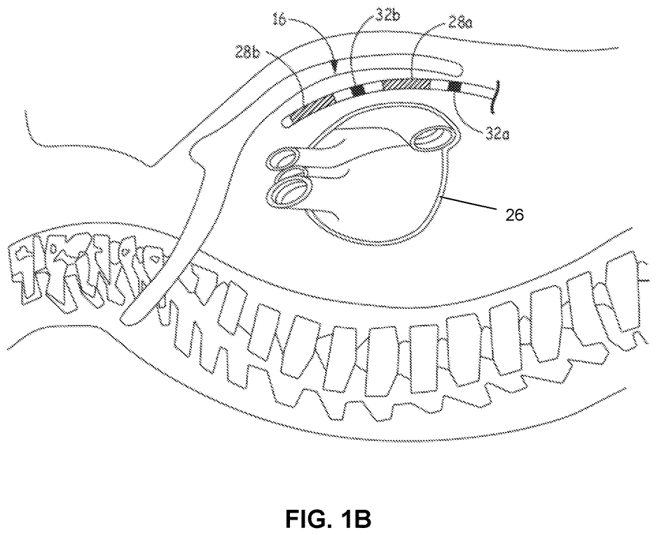

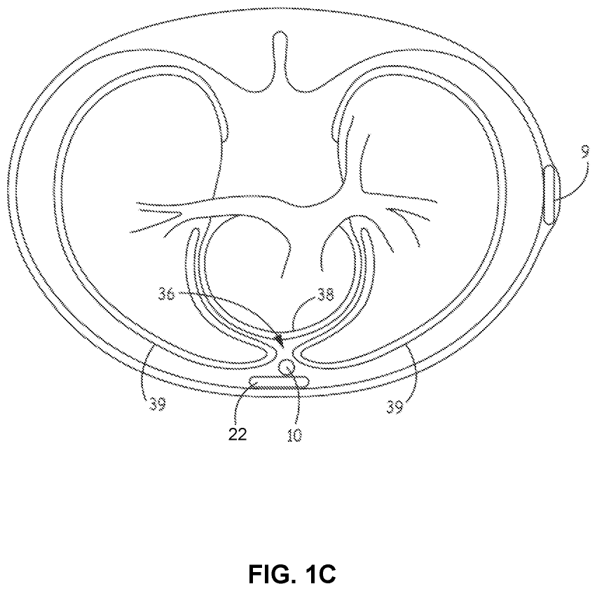

FIGS. 1A-1C are conceptual diagrams illustrating an example system 8 that may be used for sensing of physiological parameters of patient 12 and/or to provide therapy to heart 26 of patient 12. System 8 includes ICD 9, which is coupled to lead 10. While FIG. 1A-1C shows ICD 9 coupled to lead 10, ICD 9 may also be coupled to a plurality of leads. ICD 9 may be, for example, an implantable pacemaker, cardioverter, and/or defibrillator that provides electrical signals to heart 26 via electrodes coupled to lead 10. Patient 12 is ordinarily, but not necessarily a human patient.

FIG. 1A is a front view of a patient 12 implanted with extravascular ICD system 8 implanted intra-thoracically. Referring now to the drawings in which like reference designators refer to like elements, there is shown in FIGS. 1A-1C conceptual diagrams illustrating various views of an example extravascular ICD system 8. ICD system 8 includes an ICD 9 connected to an implantable medical lead 10. FIG. 1A is a front view of a patient implanted with extravascular ICD system 8. FIG. 1B is a side view of the patient implanted with extravascular ICD system 8. FIG. 1C is a transverse view of the patient implanted with extravascular ICD system 8.

ICD 9 may include a housing that forms a hermetic seal that protects components inside the ICD 9. The housing of ICD 9 may be formed of a conductive material, such as titanium or titanium alloy, which may function as a housing electrode (sometimes referred to as a can electrode). In some embodiments, ICD 9 may be formed to have or may include a plurality of electrodes on the housing. ICD 9 may also include a connector assembly 34 (also referred to as a connector block or header) that includes electrical feedthroughs through which electrical connections are made between conductors of lead 10 and electronic components included within the housing of ICD 9. As will be described in further detail herein, the housing may house one or more processors, memories, transmitters, receivers, sensors, sensing circuitry, therapy circuitry, power sources and other appropriate components. The housing is configured to be implanted in a patient, such as patient 12.

ICD 9 may be implanted extra-thoracically on the left side of the patient, e.g., under the skin and outside the ribcage (subcutaneously or submuscularly). ICD 9 may, in some instances, be implanted between the left posterior axillary line and the left anterior axillary line of the patient. ICD 9 may, however, be implanted at other extra-thoracic locations on the patient as described later.

Lead 10 may include an elongated lead body 13 having a distal portion 16 sized to be implanted in an extravascular location proximate the heart, e.g., intra-thoracically, as illustrated in FIGS. 1A-1C, or extra-thoracically. For example, lead 10 may extend extra-thoracically under the skin and outside the ribcage (e.g., subcutaneously or submuscularly) from ICD 9 toward the center of the torso of the patient, for example, toward the xiphoid process 23 of the patient. At a position proximate xiphoid process 23, the lead body 13 may bend or otherwise turn and extend superiorly. The bend may be pre-formed and/or lead body 13 may be flexible to facilitate bending. In the example illustrated in FIGS. 1A-1C, the lead body 13 extends superiorly intra-thoracically underneath the sternum, in a direction substantially parallel to the sternum.

Distal portion 16 of lead 10 may reside in a substernal location such that distal portion 16 of lead 10 extends superior along the posterior side of the sternum substantially within the anterior mediastinum 36. Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic arteries, and the internal thoracic veins (ITVs).

Lead body 13 may extend superiorly extra-thoracically (instead of intra-thoracically), e.g., either subcutaneously or submuscularly above the ribcage/sternum. Lead 10 may be implanted at other locations, such as over the sternum, offset to the right of the sternum, angled lateral from the proximal or distal end of the sternum, or the like. In some examples, lead 10 may be implanted within an extracardiac vessel within the thorax, such as the ITVs, the intercostal veins, the superior epigastric vein, or the azygos, hemiazygos, and accessory hemiazygos veins. In some examples, distal portion 16 of lead 10 may be oriented differently than is illustrated in FIGS. 1A-1C, such as orthogonal or otherwise transverse to sternum 22 and/or inferior to heart 26. In such examples, distal portion 16 of lead 10 may be at least partially within anterior mediastinum 36. In some examples, distal portion 16 of lead 10 may be placed between the heart and lung as well as within the pleural cavity.

Lead body 13 may have a generally tubular or cylindrical shape and may define a diameter of approximately 3-9 French (Fr). However, lead bodies of less than 3 Fr and more than 9 Fr may also be utilized. In another configuration, lead body 13 may have a flat, ribbon, or paddle shape with solid, woven filament, or metal mesh structure, along at least a portion of the length of the lead body 13. In such an example, the width across lead body 13 may be between 1-3.5 mm. Other lead body designs may be used without departing from the scope of this application.

Lead body 13 may be formed from a non-conductive material, including silicone, polyurethane, fluoropolymers, mixtures thereof, and other appropriate materials, and shaped to form one or more lumens (not shown), however, the techniques are not limited to such constructions. Distal portion 16 may be fabricated to be biased in a desired configuration, or alternatively, may be manipulated by the user into the desired configuration. For example, the distal portion 16 may be composed of a malleable material such that the user can manipulate the distal portion into a desired configuration where it remains until manipulated to a different configuration.

Lead body 13 may include a proximal portion 14 and a distal portion 16 which include electrodes configured to deliver electrical energy to the heart or sense electrical signals of the heart. Distal portion 16 may be anchored to a desired position within the patient, for example, substernally or subcutaneously by, for example, suturing distal portion 16 to the patient's musculature, tissue, or bone at the xiphoid process entry site. In some examples, distal portion 16 may be anchored to the patient or through the use of rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Lead body 13 may define a substantially linear portion 20 (FIG. 1A) as it curves or bends near the xiphoid process 23 and extends superiorly. In some examples, at least a part of distal portion 16 may define a three-dimensional undulating pattern, e.g., zig-zag, meandering, sinusoidal, serpentine, or other pattern, as it extends toward the distal end of lead 10.

Distal portion 16 includes one or more defibrillation electrodes configured to deliver an anti-tachyarrhythmia, e.g., cardioversion/defibrillation, shock to heart 26 of patient 12. In some examples, distal portion 16 includes a plurality of defibrillation electrodes spaced a distance apart from each other along the length of distal portion 16. In examples illustrated by FIGS. 1A-1C, distal portion 16 includes two defibrillation electrodes 28a and 28b (collectively, “defibrillation electrodes 28”).

Defibrillation electrodes 28 may be disposed around or within the lead body 13 of the distal portion 16, or alternatively, may be embedded within the wall of the lead body 13. In one configuration, defibrillation electrodes 28 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole, and other polymers. In another configuration, each of defibrillation electrodes 28 may be a flat ribbon electrode, a paddle electrode, a braided or woven electrode, a mesh electrode, a directional electrode, a patch electrode or another type of electrode configured to deliver a cardioversion/defibrillation shock to heart 26 of patient 12.

Defibrillation electrodes 28 may be electrically connected to one or more conductors, which may be disposed in the body wall of lead body 13 or in one or more insulated lumens (not shown) defined by lead body 13. In an example configuration, each of defibrillation electrodes 28 is connected to a common conductor such that a voltage may be applied simultaneously to all defibrillation electrodes 28 to deliver an anti-tachyarrhythmia shock to heart 26. In other configurations, defibrillation electrodes 28 may be attached to separate conductors such that each defibrillation electrode 28 may apply a voltage independent of the other defibrillation electrodes 28. In this case, ICD 9 or lead 10 may include one or more switches or other mechanisms to electrically connect the defibrillation electrodes together to function as a common polarity electrode such that a voltage may be applied simultaneously to all defibrillation electrodes 28 in addition to being able to independently apply a voltage.

Distal portion 16 may also include one or more pacing and/or sensing electrodes configured to deliver pacing pulses to heart 26 and/or sense electrical activity of heart 26. Such electrodes may be referred to as pacing electrodes, sensing electrodes, or pacing/sensing electrodes. In examples illustrated by FIGS. 1A-1C, distal portion 16 includes two pacing/sensing electrodes 32a and 32b (collectively, “pacing/sensing electrodes 32”).

In some examples, electrodes 32 may be configured to deliver low-voltage electrical pulses to the heart and/or may sense a cardiac electrical activity, e.g., depolarization and repolarization of the heart. As such, electrodes 32 may be referred to herein as pacing/sensing electrodes 32. In one configuration, electrodes 32 are ring electrodes. However, in other configurations electrodes 32 may be any of a number of different types of electrodes, including ring electrodes, short coil electrodes, paddle electrodes, hemispherical electrodes, electrode segments extending circumferentially around less than half of a circumference of the lead body, or directional electrodes. Each of electrodes 32 may be the same or different types of electrodes as others of electrodes 32. Electrodes 32 may be electrically isolated from an adjacent defibrillation electrode 28 by including an electrically insulating layer of material between electrodes 32 and adjacent defibrillation electrodes 28. Each electrode 32 may have its own separate conductor such that a voltage may be applied to or sensed via each electrode independently from another electrode 32.

Electrodes 28 are referred to as defibrillation electrodes, and electrodes 32 are referred to as pacing/sensing electrodes, because they may have different physical structures enabling different functionality. Defibrillation electrodes 28 may be larger, e.g., have greater surface area, than pacing/sensing electrodes 32 and, consequently, may be configured to deliver anti-tachyarrhythmia shocks that have relatively higher voltages than pacing pulses. The relatively smaller size of pacing/sensing electrodes 32 may provide advantages over defibrillation electrodes for delivering pacing pulses and sensing intrinsic cardiac activity, e.g., lower pacing capture thresholds and/or better sensed signal quality. Nevertheless, a defibrillation electrode 28 may be used to deliver pacing pulses and/or sense electrical activity of the heart, such as in combination with a pacing/sensing electrode 32. In some examples, sensing of electrical activity of the heart may be between sensing electrode 32b and all or a portion of one of defibrillation electrodes 28a, 28b.

Proximal portion 14 of lead body 13 may include one or more connectors 34 to electrically couple lead 10 to ICD 9. ICD 9 may also include a connector assembly that includes electrical feedthroughs through which electrical connections are made between the one or more connectors 34 of lead 10 and the electronic components included within the housing. The housing of ICD 9 may house one or more processors, memories, transmitters, receivers, sensors, sensing circuitry, therapy circuitry, power sources (e.g., capacitors and batteries), and/or other components. The components of ICD 9 may generate and deliver electrical therapy such as anti-tachycardia pacing, cardioversion or defibrillation shocks, post-shock pacing, and/or bradycardia pacing.

The three-dimensional undulating configuration of distal portion 16 and the inclusion of electrodes 32 between defibrillation electrodes 28 may provide a number of therapy vectors for the delivery of electrical therapy to the heart. For example, at least a portion of defibrillation electrodes 28 and one of electrodes 32 may be disposed over the right ventricle, or any chamber of the heart, such that pacing pulses and anti-tachyarrhythmia shocks may be delivered to the heart. The housing of ICD 9 may be charged with or function as a polarity different than the polarity of the one or more defibrillation electrodes 28 and/or electrodes 32 such that electrical energy may be delivered between the housing and the defibrillation electrode 28 and/or electrode 32 to the heart.

Each defibrillation electrode 28 may have the same polarity as every other defibrillation electrode 28 when a voltage is applied to it such that a shock may be delivered from all defibrillation electrodes together. In examples in which defibrillation electrodes 28 are electrically connected to a common conductor within lead body 13, this is the configuration of defibrillation electrodes 28. However, in other examples, defibrillation electrodes 28 may be coupled to separate conductors within lead body 13 and may therefore each have different polarities such that electrical energy may flow between defibrillation electrodes 28, or between one of defibrillation electrodes 28 and one of pacing/sensing electrodes 32 or the housing electrode, to provide anti-tachyarrhythmia shock, pacing therapy, and/or to sense cardiac depolarizations. In this case, defibrillation electrodes 28 may still be electrically coupled together, e.g., via one or more switches within ICD 9, to have the same polarity.

ICD 9 may be in wireless communication with another implanted medical device and/or may be in wireless communication with one or more patient computing device(s), e.g., patient computing device(s) 18. Patient computing device(s) 18 are configured for wireless communication with ICD 9. Computing device(s) 18 retrieve sensed physiological data from ICD 9 that was collected and stored by the ICD 9. In some examples, computing device(s) 18 take the form of personal computing devices of patient 12. For example, computing device(s) 18 may take the form of a smartphone of patient 12, a smartwatch or other smart apparel of patient 12. In some examples, computing device(s) 18 may be any computing device configured for wireless communication with ICD 9 such as a desktop, laptop, or tablet computer. Computing device(s) 18 may communicate with ICD 9 according to the Bluetooth® or Bluetooth® Low Energy (BLE) protocols, as examples. Computing device(s) 18 may be configured to communicate with a variety of other devices or systems via a network. For example, one or more of computing device(s) 18 may be configured to communicate with one or more computing systems that may be respectively managed by manufacturers of ICD 9 and computing device(s) 18 to, for example, provide cloud storage and analysis of collected data, maintenance and software services, or other networked functionality for their respective devices and users thereof. Computing system may comprise, or may be implemented by, the Medtronic CareLink™ Network, in some examples.

Although shown in FIG. 1 as a stand-alone device for purposes of example, computing device(s) 18 may be any component or system that includes processing circuitry or other suitable computing environment for executing software instructions and, for example, need not necessarily include one or more elements shown in FIG. 1. Computing device(s) 18 may include communication circuitry to communicate with other devices by transmitting and receiving data. Computing device(s) 18 may receive data from ICD 9, such as cardiac signals, noise, and/or interference, from communication circuitry in ICD 9. Computing device(s) may send data to ICD 9, such as data relating to noise/interference history or noise/interference episodes. Computing device(s) 18 may include a network interface card, such as an Ethernet card, an optical transceiver, a radio frequency transceiver, or any other type of device that can send and receive information. For example, computing device(s) 18 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

Computing device(s) 18 may be configured to execute an AI engine that operates according to one or more models, such as machine learning models. Machine learning models may include any number of different types of machine learning models, such as neural networks, convolutional neural networks, deep neural networks, dense neural networks, and the like. Although described with respect to machine learning models, the techniques described in this disclosure are also applicable to other types of AI models, including rule-based models, finite state machines, and the like.

Machine learning may generally enable a computing device to analyze input data and identify an action to be performed responsive to the input data. Each machine learning model may be trained using training data that reflects likely input data. The training data may be labeled or unlabeled (meaning that the correct action to be taken based on a sample of training data is explicitly stated or not explicitly stated, respectively).

The training of the machine learning model may be guided (in that a designer, such as a computer programmer, may direct the training to guide the machine learning model to identify the correct action in view of the input data) or unguided (in that the machine learning model is not guided by a designer to identify the correct action in view of the input data). In some instances, the machine learning model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

Computing device(s) 18 may utilize machine learning, such as a deep learning algorithm or model (e.g., a neural network or deep belief network), to generate a score indicative of whether noise is present in the cardiac signal and/or determine a risk score that noise will be predominately present for a period of time, such as the next hour, next day, next week, next month, next 6-months, etc. The generated score may be output as an indication of whether the risk of noise is greater than or equal to a risk threshold.

For example, a deep learning algorithm or machine learning model may be trained with clean cardiac signals and cardiac signals with different levels of noise with different frequencies (e.g., 50 Hz, 60 Hz, and 16.7 Hz, etc.). For example, 16.7 Hz corresponds to European subway system noise. The output of the deep learning algorithm or machine learning model may be a score to indicate whether noise is present or not. Accordingly, computing device 18 may be able to determine whether noise is present or not very accurately since noise may have a distinct signature which is very different than cardiac signals. The deep learning algorithm or machine learning model may also generate a score to indicate the noise will be present for a period of time, based on the score of noise existence indication, historical pattern of the noise occurrence (day, time, etc.), patient activity level, GPS location, patient's notes, etc., which may be input into the model to predict how long the noise will be present.

The deep learning algorithm or machine learning model may also generate a score to indicate the noise will be present for a period of time, based on the score of noise existence indication, historical pattern of the noise occurrence (day, time, etc.), patient activity level, GPS location, patient's notes, etc., which could be fed into the model to predict how long the noise will last.

The techniques of this disclosure may be applied to implantable systems other than ICD 9, including, but not limited to, bradycardia pacemaker systems, as well as with external stimulation devices, such as permanent or temporary external pacemakers or defibrillators.

In some examples, lead 10 may include a first defibrillation electrode 28a and a second defibrillation electrode 28b that are configured to deliver anti tachyarrhythmia shocks. In this example, pacing electrode 32b may be configured to deliver a pacing pulse that generates an electric field proximate to the pacing electrode.

As discussed above, in the examples of FIGS. 1A-1C, lead 10 may sense electrical activity of heart 26, such as by electrodes 32 that may sense a cardiac electrical activity, e.g., depolarization and repolarization of the heart, and/or deliver electrical stimulation to heart 26., such as by defibrillation electrodes 28 and/or pacing/sensing electrodes 32.

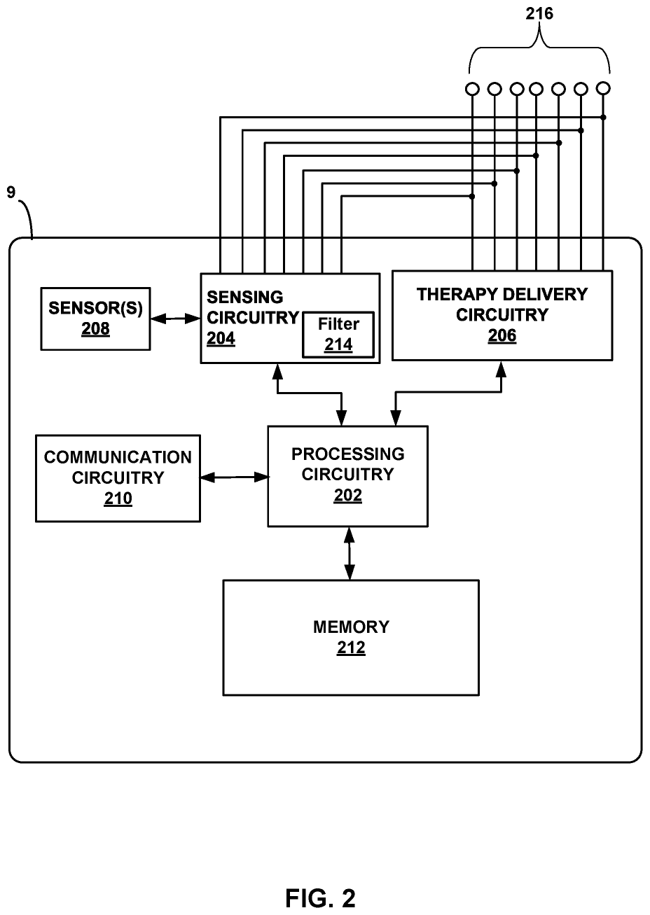

FIG. 2 is a functional block diagram of an example configuration of electronic components and other components of ICD 9. ICD 9 includes a processing circuitry 202, sensing circuitry 204, therapy delivery circuitry 206, sensors 208, communication circuitry 210, and memory 212. In some examples, ICD 9 may include more or fewer components. The described circuitry and other components may be implemented together on a common hardware component or separately as discrete but interoperable hardware or software components. Depiction of different features is intended to highlight different functional aspects and does not necessarily imply that such circuitry and other components must be realized by separate hardware or software components. Rather, functionality associated with one or more circuitries and components may be performed by separate hardware or software components, or integrated within common or separate hardware or software components.

Sensing circuitry 204 may be electrically coupled to some or all of electrodes 216, which may correspond to any of the defibrillation, pacing/sensing, and housing electrodes described herein. Sensing circuitry 204 may be coupled to some or all of sensor(s) 208. Sensing circuitry 204 is configured to obtain signals sensed via one or more combinations of electrodes 216 and/or sensor(s) 208 and process the obtained signals. Sensing circuitry 204 may include filter 214. In some examples, sensing circuitry 204 may include a plurality of filters 214. In some examples, sensing circuitry 204 may be implemented in the processing circuitry 202 of ICD 9.

The components of sensing circuitry 204 may be analog components, digital components or a combination thereof. Sensing circuitry 204 may, for example, include one or more sense amplifiers, filters, rectifiers, threshold detectors, analog-to-digital converters (ADCs) or the like. Sensing circuitry 204 may convert the sensed signals to digital form and provide the digital signals to processing circuitry 202 for processing or analysis. For example, sensing circuitry 204 may amplify signals from the sensing electrodes and convert the amplified signals to multi-bit digital signals by an ADC. Sensing circuitry 204 may also compare processed signals to a threshold to detect the existence of atrial or ventricular depolarizations (e.g., P-or R waves) and indicate the existence of the atrial depolarization (e.g., P-waves) or ventricular depolarizations (e.g., R-waves) to processing circuitry 202. As shown in FIG. 2, ICD 9 may additionally include one or more sensors 208, such as one or more accelerometers, which may be configured to provide signals indicative of other parameters of a patient, such as activity or posture, to processing circuitry 202.

Processing circuitry 202 may process the signals from sensing circuitry 204 to monitor electrical activity of heart 26 of patient 12. Processing circuitry 202 may store signals obtained by sensing circuitry 204 as well as any generated EGM waveforms, marker channel data or other data derived based on the sensed signals in memory 212. Processing circuitry 202 may analyze the EGM waveforms and/or marker channel data to detect arrhythmias (e.g., bradycardia or tachycardia).

In response to detecting a cardiac event, processing circuitry 202 may control therapy delivery circuitry 206 to deliver the desired therapy to treat the cardiac event, e.g., defibrillation shock, cardioversion shock, ATP, post shock pacing, or bradycardia pacing.

Therapy delivery circuitry 206 is configured to generate and deliver electrical therapy to heart 26. Therapy delivery circuitry 206 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 206 may include a first set of components configured to provide pacing therapy and a second set of components configured to provide defibrillation therapy. In some instances, therapy delivery circuitry 206 may utilize the same set of components to provide both pacing and defibrillation therapy. In still other instances, therapy delivery circuitry 206 may share some of the defibrillation and pacing therapy components while using other components solely for defibrillation or pacing. Processing circuitry 202 may control therapy delivery circuitry 206 to deliver the generated therapy to heart 26 via one or more combinations of electrodes 216. Although not shown in FIG. 2, ICD 9 may include switching circuitry configurable by processing circuitry 202 to control which of electrodes 216 is connected to therapy delivery circuitry 206 and sensing circuitry 204.

Processing circuitry 202 may receive from one or more sensing electrodes 32, coupled to ICD 9, one or more cardiac signals of patient 12. In some examples, the one or more sensing electrodes may be in positioned in a lead coupled to an ICD or be positioned in a leadless ICD. Processing circuitry 202 may also receive noise from one or more sensing electrodes 32. A non-limiting example of noise is electromagnetic interference (EMI). The cardiac signals may include noise and may be corrupted by the noise. The noise corrupting the cardiac signals may cause false detections of ventricular fibrillation (VF) and result in subsequent unnecessary delivery of shocks by ICD 9.

Processing circuitry 202 may determine one or more of noise risk being above a noise risk threshold and/or active noise being above an active noise threshold, and in response to determining one or more of the noise risk is greater than or equal to the noise risk threshold and/or the active noise is greater than or equal to the active noise threshold, processing circuitry 202 may activate filter 214 to filter the noise from the cardiac signal, which may result in a reduction of unnecessary delivery of shocks by ICD 9. In some examples, processing circuitry 202 may determine noise risk being above a noise risk threshold, and in response to determining the noise risk is greater than or equal to the noise risk threshold, processing circuitry 202 may activate filter 214 to filter the noise from the cardiac signal, which may result in a reduction of unnecessary delivery of shocks by ICD 9.

In some examples, a risk of noise may be indicated based on a history of noise. For example, if patient's 12 place of occupation and/or residence is at a location with strong noise signals, cardiac signals received from one or more sensing electrodes 32 would likely include the noise and be corrupted. If processing circuitry determines the risk of noise is greater than or equal to a threshold, such as a predetermined threshold that indicates the corrupted cardiac signals may result in unnecessary delivery of shocks by ICD 9, processing circuitry 202 activates filter 214 to filter noise. A risk score may indicate the risk of noise or likelihood of noise. In some examples, the threshold may be a noise to signal ratio, which is a ratio of noise level to cardiac signal level. In some examples, a ratio threshold may be selected as a value that once it is crossed the noise will be oversensed or the processing circuitry 202 may be unable to accurately classify the corrupted signals as VT or VF.

In some examples, processing circuitry 202 may determine a type of noise corresponding to the detected noise risk, such as the frequency of the noise (e.g., 50 Hz, 60 Hz, 16.7 Hz) and adjust filter 214 to filter signals corresponding to the type of noise detected. For example, when processing circuitry 202 detects the frequency of the noise to be 50 Hz, filter 214 may be adjusted to filter 50 Hz signals.

In some examples, processing circuitry 202 may initially determine a frequency spectrum of a true clean cardiac signal and use this frequency spectrum as a reference. Processing circuitry 202 may determine that frequency point(s) on a spectrum of a cardiac signal having extra energy above a threshold, compared to the reference spectrum, may be treated as noise. Processing circuitry 202 may tune filter 214 for the noise frequency and apply the tuned filter 214 to filter out the noise with that specific frequency. For example, if the cardiac signal has certain level of 60 Hz EMI noise, then, on the frequency spectrum, at 60 Hz, there may be prominent energy compared to its neighboring frequency components (e.g., the reference frequency), then, the processing circuitry 202 may determine the cardiac signal is corrupted by 60 Hz EMI. In response, processing circuitry 202 may then apply a 60 Hz filter, such as a notch filter, to filter out the noise. Similar techniques may be applied to other frequencies of EMI, such as, 16.7 Hz, 50 Hz, etc.

ICD 9 may send received cardiac signals to computing device(s) 18 which may apply the cardiac signals to a machine learning model to determine a risk of noise. In some examples, a clinician may determine the received cardiac signals include noise and that the noise risk is above a risk threshold. In some examples, noise risk may be determined based on a number of short V-V interval counts in cardiac signal over a period of time. If the number of short V-V interval counts over a period of time is above an interval count threshold, noise risk may be determined to be above a noise risk threshold.

Processing circuitry 202 may, upon activating filter 214, determine whether one or more of the noise risk becomes less than the noise risk threshold or the active noise becomes less than the active noise threshold. In response to determining one or more of the noise risk is less than the noise risk threshold or the active noise is less than the active noise threshold, processing circuitry 202 may disable filter 214. In some examples, filter 214 may have intensive current drain which may negatively impact battery life when filter 214 is activated that may decrease the longevity of ICD 9. In some examples, processing circuitry 202 may upon activating filter 214, determine whether the noise risk becomes less than the noise risk threshold. In response to determining the noise risk is less than the noise risk threshold, processing circuitry 202 may disable filter 214. Accordingly, selectively activating filter 214 in accordance with the techniques may help reduce unnecessary delivery of shocks by ICD 9 while minimizing the effect activating filter 214 has on the battery life of ICD 9.

In some examples, in response to determining the noise risk is greater than or equal to the noise risk threshold, processing circuitry 202 may activate filter 214 to be on for a period of time greater than a noise risk time threshold. The period of time may correspond to a period of time the noise risk is determined to be greater than a noise risk threshold. In some examples, processing circuitry 202 may determine a period of time the noise risk is likely to be above a noise risk threshold, such as by applying the cardiac signals to a machine learning model and receiving a period of time, generated by the machine learning model, the noise risk is to be above the noise risk threshold. Processing circuitry 202 may then activate and disable filter 214 to correspond to the period of time the determined noise risk is above the noise risk threshold.

In some examples, processing circuitry 202 may automatically disable filter 214 when the period of time ends. In some examples, in response to the period of time ending, processing circuitry 202 may compare the noise risk to the noise risk threshold. In response to the noise risk being less than the noise risk threshold, processing circuitry 202 may disable filter 214. In response to the noise risk being greater than or equal to the noise risk threshold, processing circuitry 202 may maintain filter 214 being activated.

In response to determining the active noise is greater than or equal to the active noise threshold, processing circuitry 202 may activate filter 214. Processing circuitry 202 may determine whether the active noise becomes less than the active noise threshold, and in response to the active noise becoming less than the active noise threshold, disable filter 214. In some examples, processing circuitry 202 may determine a type of noise corresponding to the detected active noise, such as the frequency of the active noise (e.g., 50 Hz, 60 Hz, 16.7 Hz) and adjust filter 214 to filter signals corresponding to the type of noise detected. For example, when processing circuitry 202 detects the frequency of the noise to be 50 Hz, filter 214 may be adjusted to filter 50 Hz signals. In some examples, filter 214 may be a notch filter.

In some examples, processing circuitry 202 may initially determine a frequency spectrum of a true clean cardiac signal and use this frequency spectrum as a reference. Processing circuitry 202 may determine that frequency point(s) on a spectrum of a cardiac signal having extra energy above a threshold, compared to the reference spectrum, may be treated as noise. Processing circuitry 202 may tune filter 214 for the noise frequency and apply the tuned filter 214 to filter out the noise with that specific frequency. For example, if the cardiac signal has certain level of 60 Hz EMI noise, then, on the frequency spectrum, at 60 Hz, there may be prominent energy compared to its neighboring frequency components (e.g., the reference frequency), then, the processing circuitry 202 may determine the cardiac signal is corrupted by 60 Hz EMI. In response, processing circuitry 202 may then apply a 60 Hz filter, such as a notch filter, to filter out the noise. Similar techniques may be applied to other frequencies of EMI, such as, 16.7 Hz, 50 Hz, etc.

Processing circuitry 202 may determine active EMI based on one or more of 1) a probable detection state of noise is identified, 2) one or more of ventricular depolarization V-V intervals or R-R intervals being less than an interval timing threshold value, or 3) noise is above a noise threshold value. In some examples, frequency of active noise may be determined using a Fast Fourier transform.

In response to detecting a cardiac event, processing circuitry 202 may control therapy delivery circuitry 206 to deliver the desired therapy to treat the cardiac event, e.g., defibrillation shock, cardioversion shock, ATP, post shock pacing, or bradycardia pacing.

Therapy delivery circuitry 206 is configured to generate and deliver electrical therapy to heart 26. Therapy delivery circuitry 206 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 206 may include a first set of components configured to provide pacing therapy and a second set of components configured to provide defibrillation therapy. In some instances, therapy delivery circuitry 206 may utilize the same set of components to provide both pacing and defibrillation therapy. In still other instances, therapy delivery circuitry 206 may share some of the defibrillation and pacing therapy components while using other components solely for defibrillation or pacing. Processing circuitry 202 may control therapy delivery circuitry 206 to deliver the generated therapy to heart 26 via one or more combinations of electrodes 216. Although not shown in FIG. 2, ICD 9 may include switching circuitry configurable by processing circuitry 202 to control which of electrodes 216 is connected to therapy delivery circuitry 206 and sensing circuitry 204.

Communication circuitry 210 may include any suitable hardware, firmware, software or any combination thereof for communicating with another device, such as a clinician programmer, a patient monitoring device, or the like. For example, communication circuitry 210 may include appropriate modulation, demodulation, frequency conversion, filtering, and amplifier components for transmission and reception of data with the aid of an antenna.

The various components of ICD 9 may include any one or more processors, controllers, digital signal processors (DSPs), application specific integrated circuits (ASICs), field-programmable gate arrays (FPGAs), or equivalent discrete or integrated circuitry, including analog circuitry, digital circuitry, or logic circuitry. Processing circuitry 202 may include fixed function circuitry and/or programmable processing circuitry. The functions attributed to processing circuitry 202 herein may be embodied as software, firmware, hardware or any combination thereof.

Memory 212 may include computer-readable instructions that, when executed by processing circuitry 202 or other components of ICD 9, cause one or more components of ICD 9 to perform various functions attributed to those components in this disclosure. Memory 212 may include any volatile, non-volatile, magnetic, optical, or electrical media, such as a random-access memory (RAM), read-only memory (ROM), non-volatile RAM (NVRAM), static non-volatile RAM (SRAM), electrically-erasable programmable ROM (EEPROM), flash memory, or any other non-transitory computer-readable storage media.

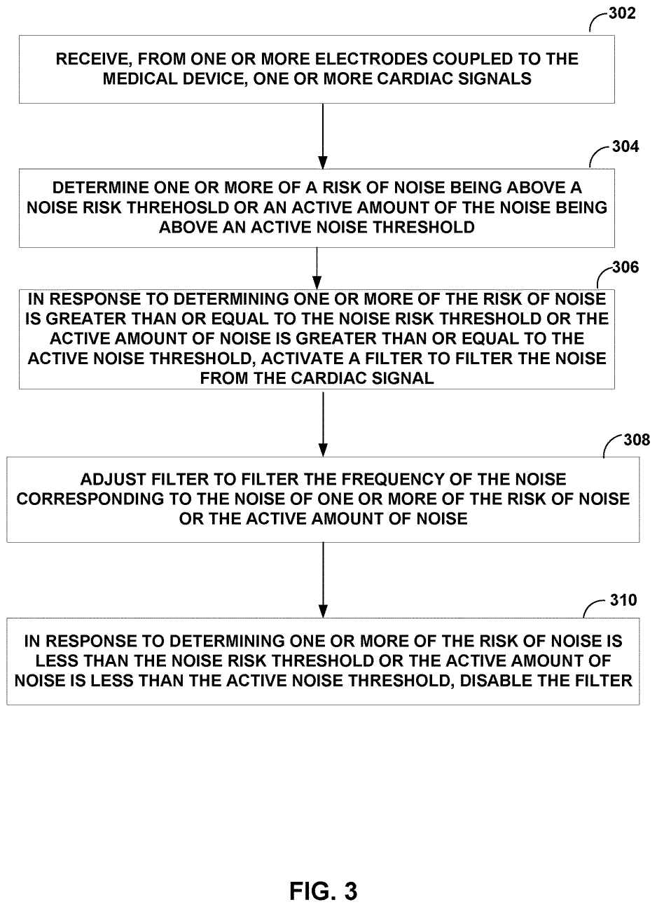

FIG. 3 is a flow diagram illustrating an example technique of activating a filter to filter EMI from a cardiac signal in accordance with ICD 9, as shown in FIG. 2. In some examples, processing circuitry 202 may receive, from one or more sensing electrodes 32 coupled to a medical device, one or more cardiac signals (302). In some examples, processing circuitry 202 may determine one or more of noise risk being greater than or equal to a noise risk threshold or active noise being greater than or equal to an active noise threshold (304). In response to determining one or more of the noise risk is greater than or equal to the noise risk threshold or the active noise is greater than or equal to the active noise threshold, processing circuitry 202 may activate filter 214 to filter the noise from the cardiac signal (306). In some examples, processing circuitry 202 may adjust filter 214 to filter the frequency of the noise corresponding to one or more of the noise of the noise risk or the active noise (308). In response to determining one or more of the noise risk is less than the risk threshold or the active noise is less than the active noise threshold, processing circuitry 202 may disable filter 214 (310).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A medical device includes a memory; and processing circuitry coupled to the memory, the processing circuitry is configured to: receive, from one or more electrodes coupled to the medical device, a cardiac signal; determine a risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activate a filter to filter the noise from the cardiac signal.

Example 2: The medical device of example 1, wherein the processing circuitry is further configured to: upon activating the filter, determine whether the risk of noise becomes less than the noise risk threshold or the active amount of noise becomes less than the active noise threshold; and in response to determining the risk of noise is less than the noise risk threshold or the active amount of noise is less than the active noise threshold, disable the filter.

Example 3: The medical device of any of examples 1 through 2, wherein the processing circuitry is further configured to: in response to determining the risk of noise being greater than or equal to the noise risk threshold, activate the filter.

Example 4: The medical device of any of examples 1 through 3, wherein the processing circuitry is further configured to: determine a frequency of noise corresponding to the risk of noise; and adjust the filter to filter the frequency of the noise corresponding to the risk of noise.

Example 5: The medical device of any of examples 1 through 4, wherein the processing circuitry is further configured to: in response to determining the risk of noise is greater than or equal to the noise risk threshold, activate the filter to be on for a period of time greater than a noise risk time threshold.

Example 6: The medical device of example 5, wherein the processing circuitry is further configured to: in response to the period of time ending, compare the risk of noise to the noise risk threshold; in response to the risk of noise being less than the noise risk threshold, disable the filter; and in response to the risk of noise being greater than or equal to the noise risk threshold, maintain the filter being activated.

Example 7: The medical device of any of examples 1 through 6, wherein the processing circuitry is further configured to: in response to determining the active amount of noise is greater than or equal to the active noise threshold, activate the filter; determine whether the active amount of noise becomes less than the active noise threshold; and in response to the active amount of noise becoming less than the active noise threshold, disable the filter.

Example 8: The medical device of any of examples 1 through 7, wherein the processing circuitry is further configured to: determine a frequency of noise corresponding to the active amount of noise; and adjust the filter to filter the frequency of the noise corresponding to the active amount of noise.

Example 9: The medical device of any of examples 1 through 8, wherein the processing circuitry determines risk of noise based on one or more of 1) one or more R-R intervals of high-rate episodes are below an interval threshold value, 2) one or more short interval counts (SIC) are greater than or equal to an SIC threshold, or 3) an AI-adjudicated classification determines an elevated risk of EMI.

Example 10: The medical device of any of examples 1 through 9, wherein the processing circuitry determines active amount of noise based on one or more of 1) a probable detection state of noise is identified, 2) one or more of ventricular depolarization V-V intervals or R-R intervals being less than an interval timing threshold value, or 3) noise is greater than or equal to a noise threshold value.

Example 11: The medical device of any of examples 1 through 10, wherein the filter is a notch filter.

Example 12: The medical device of any of examples 1 through 11, wherein the noise is electromagnetic interference (EMI).

Example 13: The medical device of any of examples 1 through 12, wherein the processing circuitry is further configured to: detect a tachyarrhythmia based on the filtered signal; and deliver a tachyarrhythmia shock in response to detecting the tachyarrhythmia.

Example 13A: The medical device of any of claims 1 through 11, further comprising therapy delivery circuitry configured to generate electrical therapy, wherein the processing circuitry is further configured to: detect a tachyarrhythmia based on the filtered signal; and control the therapy delivery circuitry to generate and deliver a tachyarrhythmia shock in response to detecting the tachyarrhythmia.

Example 13B. A medical device system comprising the medical device of any of Examples 1 through 12; and a lead that includes: a lead body having a proximal portion and a distal portion; one or more connectors at the proximal portion of the lead body and configured to electrically couple the lead to the medical device; and the one or more electrodes configured to sense the cardiac signal.

Example 14: A medical device system includes a medical device configured to sense a cardiac signal with a sensor; and processing circuitry configured to: determine a risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activate a filter to filter the noise from the cardiac signal.

Example 15: The medical device system of example 14, wherein the processing circuitry is further configured to: upon activating the filter, determine whether the risk of noise becomes less than the noise risk threshold or the active amount of noise becomes less than the active noise threshold; in response to determining the risk of noise is less than the noise risk threshold or the active amount of noise is less than the active noise threshold, disable the filter.

Example 16: The medical device system of any of examples 14 through 15, wherein the processing circuitry is further configured to: in response to determining the risk of noise being greater than or equal to the noise risk threshold, activate the filter.

Example 17: The medical device system of any of examples 14 through 16, wherein the processing circuitry is further configured to: determine a frequency of noise corresponding to the risk of noise; and adjust the filter to filter the frequency of the noise corresponding to the noise risk.

Example 18: The medical device system of examples 14 through 17, wherein the processing circuitry is further configured to: in response to determining the risk of noise is greater than or equal to the noise risk threshold, activate the filter to be on for a period of time greater than a noise risk time threshold.

Example 19: The medical device system of example 18, wherein the processing circuitry is further configured to: in response to the period of time ending, compare the noise risk to the noise risk threshold; in response to the risk of noise being less than the noise risk threshold, disable the filter; and in response to the risk of noise being greater than or equal to the noise risk threshold, maintain the filter being activated.

Example 20: The medical device system of any of examples 14 through 19, wherein the processing circuitry is further configured to: in response to determining the active amount of noise is greater than or equal to the active noise threshold, activate the filter; determine whether the active amount of noise becomes less than the active noise threshold; and in response to the active amount of the noise becoming less than the active noise threshold, disable the filter.

Example 21: The medical device system of any of examples 14 through 20, wherein the processing circuitry is further configured to: determine a frequency of noise corresponding to the active amount of noise; and adjust the filter to filter the frequency of the noise corresponding to the active amount of noise.

Example 22: The medical device system of any of examples 14 through 21, wherein the processing circuitry determines risk of noise based on one or more of 1) one or more R-R intervals of high-rate episodes are below an interval threshold value, 2) one or more short interval counts (SIC) are greater than or equal to an SIC threshold, or 3) an AI-adjudicated classification determines an elevated risk of noise.

Example 23: The medical device system of any of examples 14 through 22, wherein the processing circuitry determines active amount of noise based on one or more of 1) a probable detection state of noise is identified, 2) one or more of ventricular depolarization V-V intervals or R-R intervals being less than an interval timing threshold value, or 3) noise is greater than or equal to a noise threshold value.

Example 24: The medical device system of any of examples 14 through 23, wherein the filter is a notch filter.

Example 25: The medical device system of any of examples 14 through 24, wherein the noise is electromagnetic interference (EMI).

Example 26: The medical device system of any of examples 14 through 25, the processing circuitry is further configured to: detect a tachyarrhythmia based on the filtered signal; and deliver a tachyarrhythmia shock in response to detecting the tachyarrhythmia.

Example 27: A method includes receiving, from one or more electrodes coupled to a medical device, a cardiac signal; determining, by processing circuitry, risk of noise being greater than or equal to a noise risk threshold or an active amount of the noise being greater than or equal to an active noise threshold; and in response to determining the risk of noise is greater than or equal to the noise risk threshold or the active amount of noise is greater than or equal to the active noise threshold, activating, by the processing circuitry, a filter to filter the noise from the cardiac signal.

Example 28: The method of example 27, the method further includes upon activating the filter, determining, by the processing circuitry, whether the risk of noise becomes less than the noise risk threshold or the active amount of noise becomes less than the active noise threshold; in response to determining the risk of noise is less than the noise risk threshold or the active amount of noise is less than the active noise threshold, disabling, by the processing circuitry, the filter.

Example 29: The method any of examples 27 through 28, the method further includes in response to determining the risk of noise being greater than or equal to the noise risk threshold, activating, by the processing circuitry, the filter.

Example 30: The method of any of examples 27 through 29, the method further includes determining, by the processing circuitry, a frequency of noise corresponding to the risk of noise; and adjusting, by the processing circuitry, the filter to filter the frequency of the noise corresponding to the risk of noise.

Example 31: The method of any of examples 27 through 30, the method further includes in response to determining the risk of noise is greater than or equal to the noise risk threshold, activating, by the processing circuitry, the filter to be on for a period of time greater than a noise risk time threshold.

Example 32: The method of example 31, the method further includes in response to the period of time ending, comparing, by the processing circuitry, the risk of noise to the noise risk threshold; in response to the risk of noise being less than the noise risk threshold, disabling, by the processing circuitry, the filter; and in response to the risk of noise being greater than or equal to the noise risk threshold, maintaining, by the processing circuitry, the filter being activated.

Example 33: The method of any of examples 27 through 32, the method further includes in response to determining the active amount of noise is greater than or equal to the active noise threshold, activating, by the processing circuitry, the filter; determining, by the processing circuitry, whether the active amount of noise becomes less than the active noise threshold; and in response to the active amount of noise becoming less than the active noise threshold, disabling, by the processing circuitry, the filter.

Example 34: The method of any of examples 27 through 33, the method further includes determining a frequency of noise corresponding to the active amount of noise; and adjusting the filter to filter the frequency of the noise corresponding to the active amount of noise.

Example 35: The method of any of examples 27 through 34, wherein the determined risk of noise is based on one or more of 1) one or more R-R intervals of high-rate episodes are below an interval threshold value, 2) one or more short interval counts (SIC) are greater than or equal to an SIC threshold, or 3) an AI-adjudicated classification determines an elevated risk of noise.

Example 36: The method of any of examples 27 through 35, wherein the determined active amount of noise is based on one or more of 1) a probable detection state of noise is identified, 2) one or more of ventricular depolarization V-V intervals or R-R intervals being less than an interval timing threshold value, or 3) noise is greater than or equal to a noise threshold value.

Example 37: The method of any of examples 27 through 36, wherein the filter is a notch filter.

Example 38: The method of any of examples 27 through 37, wherein the noise is electromagnetic interference (EMI).

Example 39: The method of any of examples 27 through 38, the method further includes detect a tachyarrhythmia based on the filtered signal; and deliver a tachyarrhythmia shock in response to detecting the tachyarrhythmia.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

1. A medical device comprising:
a memory; and
processing circuitry coupled to the memory, the processing circuitry is configured to:
receive, from one or more electrodes coupled to the medical device, a cardiac signal;
determine a risk of noise being greater than or equal to a noise risk threshold or an active amount of the noise being greater than or equal to an active noise threshold; and
in response to determining the risk of noise is greater than or equal to the noise risk threshold or the active amount of noise is greater than or equal to the active noise threshold, activate a filter to filter the noise from the cardiac signal.

2. The medical device of claim 1, wherein the processing circuitry is further configured to:
upon activating the filter, determine whether the risk of noise becomes less than the noise risk threshold or the active amount of noise becomes less than the active noise threshold; and
in response to determining the risk of noise is less than the noise risk threshold or the active amount of noise is less than the active noise threshold, disable the filter.

3. The medical device of claim 1, wherein the processing circuitry is further configured to:
determine a frequency of noise corresponding to the risk of noise; and
adjust the filter to filter the frequency of the noise corresponding to the risk of noise.

4. The medical device of any of claim 1, wherein the processing circuitry is further configured to:
in response to determining the risk of noise is greater than or equal to the noise risk threshold, activate the filter to be on for a period of time greater than a noise risk time threshold.

5. The medical device of claim 4, wherein the processing circuitry is further configured to:
in response to the period of time ending, compare the risk of noise to the noise risk threshold;
in response to the risk of noise being less than the noise risk threshold, disable the filter; and
in response to the risk of noise being greater than or equal to the noise risk threshold, maintain the filter being activated.

6. The medical device of claim 1, wherein the processing circuitry is further configured to:
in response to determining the active amount of noise is greater than or equal to the active noise threshold, activate the filter;
determine whether the active amount of noise becomes less than the active noise threshold; and
in response to the active amount of noise becoming less than the active noise threshold, disable the filter.

7. The medical device of claim 1. wherein the processing circuitry is further configured to:
determine a frequency of noise corresponding to the active amount of noise; and
adjust the filter to filter the frequency of the noise corresponding to the active amount of noise.

8. The medical device of claim 1, wherein the processing circuitry determines risk of noise based on one or more of 1) one or more R-R intervals of high-rate episodes are below an interval threshold value, 2) one or more short interval counts (SIC) are greater than or equal to an SIC threshold, or 3) an AI-adjudicated classification determines an elevated risk of electromagnetic interference (EMI).

9. The medical device of claim 1, wherein the processing circuitry determines active amount of noise based on one or more of 1) a probable detection state of noise is identified, 2) one or more of ventricular depolarization V-V intervals or R-R intervals being less than an interval timing threshold value, or 3) noise is greater than or equal to a noise threshold value.

10. The medical device of claim 1, wherein the filter is a notch filter.

11. The medical device of claim 1, wherein the noise is electromagnetic interference (EMI).

12. The medical device of claim 1, further comprising therapy delivery circuitry configured to generate electrical therapy, wherein the processing circuitry is further configured to:
detect a tachyarrhythmia based on the filtered signal; and
control the therapy delivery circuitry to generate and deliver a tachyarrhythmia shock in response to detecting the tachyarrhythmia.

13. A medical device system comprising:
the medical device of claim 1, and
a lead that includes:
a lead body having a proximal portion and a distal portion;
one or more connectors at the proximal portion of the lead body and configured to electrically couple the lead to the medical device; and
the one or more electrodes configured to sense the cardiac signal.

14. A method comprising:
receiving a cardiac signal;
determining a risk of noise being greater than or equal to a noise risk threshold or an active amount of the noise being greater than or equal to an active noise threshold; and
in response to determining the risk of noise is greater than or equal to the noise risk threshold or the active amount of noise is greater than or equal to the active noise threshold, activating a filter to filter the noise from the cardiac signal.

15. The method of claim 14, the method further comprising:
upon activating the filter, determining, by the processing circuitry, whether the risk of noise becomes less than the noise risk threshold or the active amount of noise becomes less than the active noise threshold;
in response to determining the risk of noise is less than the noise risk threshold or the active amount of noise is less than the active noise threshold, disabling, by the processing circuitry, the filter.

16. The method of claim 14, the method further comprising:
determining, by the processing circuitry, a frequency of noise corresponding to the risk of noise; and
adjusting, by the processing circuitry, the filter to filter the frequency of the noise corresponding to the risk of noise.

17. The method of claim 14, the method further comprising:
in response to determining the risk of noise is greater than or equal to the noise risk threshold, activating, by the processing circuitry, the filter to be on for a period of time greater than a noise risk time threshold.

18. The method of claim 17, the method further comprising:
in response to the period of time ending, comparing, by the processing circuitry, the risk of noise to the noise risk threshold;
in response to the risk of noise being less than the noise risk threshold, disabling, by the processing circuitry, the filter; and
in response to the risk of noise being greater than or equal to the noise risk threshold, maintaining, by the processing circuitry, the filter being activated.

19. The method of claim 14, the method further comprising:
in response to determining the active amount of noise is greater than or equal to the active noise threshold, activating, by the processing circuitry, the filter;
determining, by the processing circuitry, whether the active amount of noise becomes less than the active noise threshold; and
in response to the active amount of noise becoming less than the active noise threshold, disabling, by the processing circuitry, the filter.

20. A computer-readable medium comprising instructions that, when executed, causes processing circuitry of a medical system to:
receive, from a medical device, a cardiac signal;
determine a risk of noise being greater than or equal to a noise risk threshold or an active amount of the noise being greater than or equal to an active noise threshold; and
in response to determining the risk of noise is greater than or equal to the noise risk threshold or the active amount of noise is greater than or equal to the active noise threshold, activate a filter to filter the noise from the cardiac signal.
